(19) United States
(12) Patent Application Publication
(10) Pub. No.: US 2009/0131538 A1
(43) Pub. Date: May 21, 2009

(54) Novel propofol composition comprising a pharmaceutically acceptable salt of formaldehyde sulfoxylate

(75) Inventors: Sandhya Goyal, Rye Brook, NY (US); Suresh Dixit, Fort Worth, TX (US); Frederick Okech, Newburgh, NY (US)

Correspondence Address: VENABLE LLP, P.O. BOX 34385, WASHINGTON, DC, 20043-9998, US

(73) Assignee: Taro Pharmaceuticals North America, Inc. c/o Taro Pharmaceuticals U.S.A., Inc., Hawthorne, NY (US)

(21) Appl. No.: 11/921,101

(22) PCT Filed: May 26, 2006
(86) PCT No.: PCT/US06/20650

§ 371 Date: Feb. 11, 2008

Related U.S. Application Data
(60) Provisional application No. 60/685,679, filed on May 27, 2005.
(60) Provisional application No. 60/688,640, filed on Jun. 8, 2005.

(51) Int. Cl. A61K 31/05; A61P 23/00

(52) U.S. Cl. 514/731

(57) ABSTRACT

Sterile pharmaceutical compositions for parenteral administration containing 2,6-diisopropylphenol (propofol) are described for use as anesthetics. The compositions comprise an oil-in-water emulsion of propofol additionally comprising an amount of a pharmaceutically acceptable salt of formaldehyde sulfoxylate sufficient to prevent significant growth of microorganisms for at least 24 hours after adventitious contamination.

### CROSS REFERENCE TO RELATED APPLICATIONS

This application claims the benefit under 35 U.S.C. § 119(e) of the U.S. Provisional Application Nos. 60/685,679 filed May 27, 2005 and 60/688,640 filed Jun. 8, 2005, the disclosures of which are incorporated by reference in its entirety herein.

### FIELD OF THE INVENTION

The present invention relates to a novel sterile pharmaceutical composition for parenteral administration containing propofol and a pharmaceutically acceptable salt of formaldehyde sulfoxylate. The composition comprises an oil-in-water emulsion of propofol additionally comprising a pharmaceutically acceptable salt of formaldehyde sulfoxylate in a sufficient amount to prevent significant growth of microorganisms for at least 24 hours after adventitious contamination. The present invention also relates to the use of the composition to induce anesthesia in mammals, including sedation, and the induction and maintenance of general anesthesia.

### BACKGROUND OF THE INVENTION

Propofol (2,6-diisopropylphenol) is a widely-used, injectable anesthetic with hypnotic properties used both as a sedative, and to induce and maintain general anesthesia. Two propofol anesthetic formulations are commercially available in the US. Propofol is sold as DIPRIVAN® (trademark Zeneca) for human use as an anesthetic formulation and RAPINOVET® (trademark Zeneca) for veterinary use (e.g., dogs). Propofol is also sold as “Propofol Injectable Emulsion” (Sicor) for human use. Because the onset of anesthesia is largely controlled by a drug's diffusion rate through the blood-brain barrier, propofol's lipophilicity is key to its rapid activity. This lipophilicity, however, renders propofol relatively insoluble in water, hence it must be administered in conjunction with solubilizing agents, surfactants, or solvents; or as oil-in-water emulsions (Jones et al. (1998); U.S. Pat. No. 5,714,520). These propofol formulations contain a phospholipid, such as egg lecithin, which functions as an emulsifying agent.

Because phospholipids are good substrates for bacterial growth, non-preserved propofol oil-in-water emulsion formulations have significant drawbacks arising from the fact that these formulations support microbial growth. Propofol is often administered directly into the bloodstream either by bolus injection or by infusion. Despite handling recommendations which include immediate administration after vial entry, and disposal of infusion assemblies and of unused material after 12 hours, reports of nosocomial infections resulting from adventitious contamination are common (Bennett et al. (1995) N. Engl. J. Med. 333:147-154). Improper handling techniques include delayed administration after transfer from vial to syringe and storage for an extended time period. Preservation and sterility of propofol formulations are particularly critical.

Phospholipids are also incompatible with numerous preservatives that are at least somewhat water soluble, such as benzyl alcohol. The addition of such a preservative to a formulation containing phospholipids could destroy the formulation. Without a preservative in the formulation, any excess formulation must be thrown away within a few hours of its first use.

To overcome the contamination deficiencies found with propofol formulations, preservatives often are added in the oil-in-water formulation to preserve its sterility. U.S. Pat. Nos. 6,140,520, 5,731,355 and 5,731,356 disclose the use of EDTA in an amount sufficient to prevent no more than a 10-fold increase in microbial growth over 24 hours after adventitious extrinsic contamination with the microorganisms Staphylococcus aureus (ATCC 6538), Escherichia coli (ATCC 8739), Pseudomonas aeruginosa (ATCC 9027) and Candida albicans (ATCC 10231). A propofol preparation for clinical use is commercially available as DIPRIVAN® 1% Injection. In this formulation, a chelating or sequestering agent, (i.e., ethylene diaminetetraacetic acid (EDTA)) is included in the propofol preparation. This preparation contains propofol dissolved in soybean oil as an emulsion stabilized with egg lecithin in water. Each milliliter of this formulation consists of 10 mg/mL of propofol, 100 mg/mL of soybean oil, 22.5 mg/mL of glycerol, 12 mg/mL of egg lecithin, and disodium edetate (0.005%). Unfortunately, formulations containing EDTA is not truly an antimicrobially preserved product under USP standards as exemplified in Sklar, G. E. (1997) “Propofol and Postoperative Infections,” Ann Pharmacother, 31, 1521-3. Incidences of serious infection in human subjects have been linked to the use of DIPRIVAN®. See for example, “Bacterial Contamination of an Anesthetic Agent,” New Eng. J. Med., 333(3), 184-185; and “Microbial Growth and Endotoxin Production in the Intravenous Anesthetic Propofol,” Inf. Control Hosp. Epidem., 12(9), 535-539.

In addition, DIPRIVAN® can exhibit a thrombogenic potential in clinical use. Symptoms span the range of thrombosis and phlebitis and include incidences of burning, stinging or sensations of pain (See, Physicians Desk Reference 1999, page 3416). Rapid intravenous administration of sodium EDTA may cause hypocalcemic tetany (See, Goodman & Gilman's “The Pharmacological Basis of Therapeutics”, Tenth Edition, p. 1868).

U.S. Pat. No. 6,150,423 discloses using benzyl alcohol as preservative against microbial growth. U.S. Pat. No. 6,140,374 discloses the use of a number of antimicrobial agents in propofol containing oil-in-water emulsions including combinations of edetate and benzyl alcohol. However, addition of benzyl alcohol destroys the oil-in-water emulsion and therefore its use is restricted to formulation having a substantially phospholipid-free emulsifying agent.

U.S. Pat. No. 6,147,122 discloses a sterile oil-in-water emulsion of propofol and an amount of sodium metabisulfite. The amount of sodium metabisulfite in propofol administrated to patients requires careful monitoring not to exceed the limit set by the World Health Organization (WHO) (7.0 mg/kg as SO₂) and the amount infused in total-parenteral-nutrition amino acid formulations, as well as during peritoneal dialysis (Gunnison and Jacobsen (1987) Crit. Rev. Toxicol. 17:185-214). In addition, sodium metabisulfite is known for its potential allergy and hypersensitivity in some patients.

U.S. Pat. No. 6,028,108 discloses a sterile oil-in-water emulsion of propofol and an amount of pentetate sufficient to prevent significant growth of microorganisms for at least 24 hours after adventitious extrinsic contamination. U.S. Pat. No. 6,177,477 discloses a sterile oil-in-water emulsion of propofol and an amount of tromethamine (TRIS) sufficient to prevent significant growth of microorganisms for at least 24 hours after adventitious extrinsic contamination.

There is a continuing need to find a suitable preservative for use in the oil-in-water emulsion containing propofol. We surprisingly discovered inclusion of an amount of a pharmaceutically acceptable salt of formaldehyde sulfoxylate in a propofol oil-in-water emulsion is highly effective in preventing significant growth of a wide range of different microorganisms, including Gram (+) and Gram (−) bacteria as well as yeast and fungi, for at least 24 hours after adventitious contamination.

### SUMMARY OF THE INVENTION

The present applicants conducted an extensive and vigorous evaluation of an effective antimicrobial agent for propofol parenteral composition. The present applicants surprisingly and unexpectedly discovered that a pharmaceutically acceptable salt of formaldehyde sulfoxylate can be included in an oil-in-water emulsion of propofol and such propofol composition exerts high effectiveness in retarding or suppressing the of growth of likely microbial contaminants, without destabilizing the emulsion and without adversely reacting with other formulation components. These results are especially surprising and unexpected in light of numerous reports citing failure of attempts (i.e., many agents are reported completely ineffective) in controlling the bacterial contamination problems in parenteral formulation without the use of harmful antimicrobial agents.

The present invention includes formaldehyde sulfoxylate. The present invention also includes a pharmaceutically acceptable salt of formaldehyde sulfoxylate and combinations thereof. The pharmaceutically acceptable salt of formaldehyde sulfoxylate may include, but is not limited to, salts of sodium, potassium and the like.

Accordingly, the present invention provides a sterile composition for parenteral administration comprising an oil-in-water emulsion in which propofol is dissolved in a water-immiscible solvent that is emulsified with water wherein said emulsion is stabilized by means of a surfactant. The composition further comprises an amount of a pharmaceutically acceptable salt of formaldehyde sulfoxylate sufficient to exhibit antimicrobial activity against microorganisms most likely to contaminate the propofol preparation.

The present invention also includes the use of a pharmaceutically acceptable salt of formaldehyde sulfoxylate as a preservative for any sterile, parenterally administered oil-in-water emulsion. In addition to propofol compositions, such formulations include total-parenteral-nutrition formulations, or oil-in-water vehicles for other pharmaceutical or therapeutic agents.

### DETAILED DESCRIPTION OF THE INVENTION

### Definitions

In accordance with the present invention and as used herein, the following terms are defined to have the following meanings, unless explicitly stated otherwise:

Unless otherwise indicated, as expressed in the present specification as well as in the set of claims as % weight refers to % wt/wt. % weight refers to percentage of the weight of the referenced compound as compared to the total weight of the composition. For example, 0.05% weight formaldehyde sulfoxylate refers to % of 0.05 gram formaldehyde sulfoxylate present in a 100 gram oil-in-water propofol emulsion. For purposes of the present invention, the term “about” encompasses +/−10% of a value.

The term “emulsion” refers to a system consists of a liquid dispersed with or without an emulsifying agent in an immiscible liquid.

The term “oil-in-water emulsion” refers to a distinct two-phase system that is in equilibrium and in effect, as a whole, is kinetically stable and thermodynamically unstable.

The term “preservative” refers to an agent or agents that suppress or prevent microbiological growth at 24 hours to no more than 10-fold compared to time-zero.

The term “pharmaceutically acceptable salt” refers to all pharmaceutically acceptable salts of formaldehyde sulfoxylate. The pharmaceutically acceptable salts include sodium formaldehyde sulfoxylate or potassium formaldehyde sulfoxylate and the like that can function as a preservative in suppressing or preventing microbiological growth at 24 hours by no more than 10-fold compared to time-zero.

The term “dispersing” refers to distributing (as fine particle) of a substance evenly through a medium.

The term “water-immiscible solvent” refers to a solvent that, when mixed with water, does not form a homogeneous solution (i.e., incapable of attaining homogeneity). An exemplary water-immiscible solvent is vegetable oil.

The term “homogenizing” refers to breaking up of oil globules into very fine droplets, especially by forcing through minute openings.

The term “surfactant” refers to a surface-active agent. An example of surfactant is egg-yolk phosphatide.

The term “emulsifying agent” refers a surface-active agent promoting the formation and stabilization of an emulsion.

The present inventors surprisingly found that inclusion of a pharmaceutically acceptable salt of formaldehyde sulfoxylate in the propofol parenteral formulation is effective in suppressing or retarding bacterial growth. It is known that formaldehyde sulfoxylate is an antioxidant; however, its ability to act directly as an antimicrobial is not clear. Oil-in-water emulsions are typically formulated at pH 5.0-8.0 to assure the ionization of the headgroups of the phospholipid surfactants incorporated therein. The resulting electrostatic repulsion favors the formation of small oil particles and discourages their coalescence with time.

The present inventors have further discovered stable emulsions containing a pharmaceutically acceptable salt of formaldehyde sulfoxylate in the 5.0-8.0 pH range exhibit a good antimicrobial activity.

The present inventors have also discovered a process for the manufacture of these emulsions which minimizes the loss of the pharmaceutically acceptable salt of formaldehyde sulfoxylate as well as other ingredients in the propofol formulation.

I. Pharmaceutical Compositions

The composition of the present invention comprises a pharmaceutically acceptable salt of formaldehyde sulfoxylate. It is to be understood that the term formaldehyde sulfoxylate is intended to encompass its biologically active equivalents. A pharmaceutically acceptable salt of formaldehyde sulfoxylate (such as sodium formaldehyde sulfoxylate) may be conveniently purchased from Acros Organics (New Jersey, USA).

Collectively, the prior art fails to teach or suggest that a pharmaceutically acceptable salt of formaldehyde sulfoxylate may exert direct bactericidal or bacteriostatic effects on bacteria. Indeed, to the best of the present inventors' knowledge, a pharmaceutically acceptable salt of formaldehyde sulfoxylate has never been used as an antibacterial preservative in pharmaceutical preparations, let alone parenteral formulations. Our discovery that a pharmaceutically acceptable salt of formaldehyde sulfoxylate can exert antimicrobial activity against a broad spectrum of microorganisms is surprising.

Without wishing to be bound by theory, it is believed that a pharmaceutically acceptable salt of formaldehyde sulfoxylate may exert its antimicrobial effects via its bactericidal and bacteriostatic effects on microorganisms. The antimicrobial effects may relate to its anti-oxidant activity; but other mechanism(s) may also be involved.

A pharmaceutically acceptable salt of formaldehyde sulfoxylate will typically be present from about 0.03% to about 0.1% weight. Preferably, the pharmaceutically acceptable salt of formaldehyde sulfoxylate is present at about 0.05% weight.

A pharmaceutically acceptable salt of formaldehyde sulfoxylate includes, but not limited to, mono or divalent metal ion salt of formaldehyde sulfoxylate. Suitable metal ion salts of formaldehyde sulfoxylate include, but not limited to, sodium formaldehyde sulfoxylate, potassium formaldehyde sulfoxylate, either alone or a mixture thereof.

The composition of the present invention typically comprises about 0.1% to about 5% weight propofol. Preferable compositions comprise from about 1% to about 2% weight propofol. More preferable compositions are about 1% weight and about 2% weight propofol.

The propofol may be dissolved in a pharmaceutically acceptable water-immiscible solvent and emulsified in water and said emulsion stabilized by means of a surfactant; or the propofol may itself be emulsified in water without addition of a water-immiscible solvent and said emulsion stabilized by means of a surfactant.

Typical dosages of propofol for parenteral administration are 0.3-3 mg/kg/h, but may range to 10 mg/kg/h in exceptional cases, which is equivalent to 1.68 L emulsion/day/70 kg.

Water-immiscible solvents suitable for the preparation of oil-in-water emulsions suitable for parenteral administration are known to those skilled in the pharmaceutical arts (Handbook of Pharmaceutical Excipients Wade and Weller, Eds. (1994), American Pharmaceutical Association, The Pharmaceutical Press: London, pp 451-453). Typically, the water-immiscible solvent will be a vegetable oil: for example, soybean, safflower, cottonseed, corn, sunflower, arachis, and castor. The water-immiscible solvent may also be a wholly or partially manufactured material, for example mono-, di-, and triglycerides, fatty acid esters, or chemically and/or physically modified vegetable oils. The present invention may also comprise any combination of said water-immiscible solvents. When used, the water-insoluble solvent comprises up to about 30% weight of the composition, preferably in the range of about 5% to about 25% weight, more preferably in the range of about 10% to about 20% weight, most preferably about 10% weight.

The composition of the present invention comprises a pharmaceutically acceptable surfactant which aids in the emulsification of the water-immiscible phase in water and stabilizes said emulsion. Suitable surfactants include naturally occurring surfactants: for example, egg or soy phosphatides, either in their native or modified forms; manufactured non-ionic surfactants, for example a polyethylene glycol or esters thereof; or any mixture thereof. Preferable surfactants are egg or soy phosphatides, for example egg-yolk phospholipid. The amount of surfactant effective in producing and maintaining a stable oil-in-water emulsion will depend on the particular formulation. The factors and their relationships are well known to skilled practitioners in the pharmaceutical arts. These factors include the presence or absence of a water-immiscible solvent, the particular water-immiscible solvent used, the particular surfactant employed, the presence of salts, and the pH of the composition.

The composition of the present invention is formulated with pH in the range of about 5.0 to about 8.0. The pH may be adjusted as required by means of addition of an alkali, for example sodium hydroxide, or an acid, for example hydrochloric acid.

The composition of the present invention may be made isotonic with blood by incorporation of a suitable tonicity modifier, for example glycerin.

The compositions of the present invention are sterile, aqueous formulations and are prepared by standard manufacturing techniques using, for example, aseptic manufacturing methods and sterilization by autoclaving.

The present invention is illustrated by means of the following examples representative of the pharmaceutical formulations included in the present invention, which should not be considered as restrictions of the scope of the same.

### EXPERIMENTS

### Example 1

The present composition containing formaldehyde sulfoxylate or its pharmaceutically acceptable salts thereof was formulated to match commercial formulations in clinical performance and physical properties. Table 1 compares the present propofol composition with propofol containing EDTA (DIPRIVAN®), and propofol containing sodium metabisulfite (Propofol Injectable Emulsion). Both preparations of DIPRIVAN® and “Propofol Injectable Emulsion” were purchased commercially.

TABLE 1
Comparison of Ingredients of Present Propofol Composition
with Two Commercial Propofol Formulations

| | | | Propofol |
|---|---|---|---|
| | | Propofol | Sodium |
| | | Sodium | formaldehyde |
| | Propofol EDTA | Metabisulfite | sulfoxylate |
| | Injectable | Injectable | Injectable |
| Component | Emulsion 1% | Emulsion 1% | Emulsion 1% |
| Propofol, mg/mL | 10 | 10 | 10 |
| Soybean oil, mg/mL | 100 | 100 | 100 |
| Glycerin, mg/mL | 22.5 | 22.5 | 22.5 |
| Egg - yolk phospholipid, | 12 | 12 | 12 |
| mg/mL | | | |
| Disodium edetate, mg/mL | 0.05 | — | — |
| Sodium metabisulfite, | — | 0.25 | — |
| mg/mL | | | |
| Sodium formaldehyde | — | — | 0.5 |
| sulfoxylate, mg/mL | | | |
| WFI q.s. to 1 ml | | | |
| PH | 7.0-8.5 | 4.5-6.4 | 5.0-8.0 |

### Example 2

We compared the physical properties of the present composition with propofol containing EDTA (DIPRIVAN®), and propofol containing sodium metabisulfite. As shown in Table 2, the present composition containing a pharmaceutically acceptable salt of formaldehyde sulfoxylate (i.e., sodium formaldehyde sulfoxylate) shares similar, if not identical, many physico-chemical parameters including appearance, density, osmolality and viscosity with commercial formulations.

TABLE 2
Comparison of Physical Properties of Propofol Composition
of Present Invention With Other Commercial Formulations

| | | | Propofol |
|---|---|---|---|
| | | Propofol Sodium | sodium |
| | Propfol EDTA | Metabisulfite | formaldehyde |
| Physico- | Injectable | Injectable | sulfoxylate |
| chemical | Emulsion | Emulsion | Injectable |
| Parameter | 1.0% | 1.0% | Emulsion 1.0% |
| Appearance | White emulsion | White emulsion | White emulsion |
| | with no | with no | with no |
| | visible oil | visible oil | visible oil |
| | droplets | droplets | droplets |
| Density | 0.995 | 0.995 | 0.994 |
| Osmolality, | 300 | 300 | 313 |
| mg/ml | | | |
| Viscosity, | 1.6-1.7 | 1.6 | 1.58 |
| centistokes | | | |

The compositions of the present invention are useful as anesthetics including sedation, and induction and maintenance of general anesthesia. Thus, in another aspect, the present invention provides a method for inducing anesthesia in mammals which comprises parenteral administration of a sterile, aqueous pharmaceutical composition comprising an oil-in-water emulsion in which propofol, either alone or dissolved in a water-immiscible solvent, is emulsified in water, wherein said emulsion is stabilized by means of a surfactant; which further comprises an effective amount of formaldehyde sulfoxylate or its pharmaceutically acceptable salts thereof.

Dosage levels appropriate for the induction of desired degree of anesthesia, for example sedation, or induction of or maintenance of general anesthesia, by the compositions of the present invention will depend on the type of mammal under treatment and the physical characteristics of the specific mammal under consideration. These factors and their relationship in determining this amount are well known to skilled practitioners in the medical arts. Approximate dosage levels may be derived from the substantial literature on propofol, may be tailored to achieve optimal efficiency, and will be contingent on myriad factors recognized by those skilled in the medical arts including weight, diet, and concurrent medication.

The antimicrobial effects of formaldehyde sulfoxylate or its pharmaceutically acceptable salts thereof may also be advantageously applied to other sterile, oil-in-water emulsions for parenteral administration. Examples include total-parenteral-nutrition formulations and oil-in-water emulsions of other pharmaceuticals or therapeutic agents.

Oil-in-water emulsion including total-parenteral-nutrition formulations are administered by infusion to patients for whom oral nutrition is impossible, undesirable, or insufficient. The emulsified lipids provide a concentrated caloric content. These formulations may also contain other nutrients, for example amino acids, vitamins, and minerals. Commercial examples of such formulations include INTRALIPID® (trademark Pharmacia), LIPOFUNDINO® (trademark Braun), and TRAVAMULSION® (trademark Baxter). Accordingly, the present invention provides a sterile total-parenteral-nutrition formulation comprising lipids or fats emulsified in water which further comprises an effective amount of formaldehyde sulfoxylate or its pharmaceutically acceptable salts thereof as a preservative.

A wide variety of current and potential pharmaceutical or therapeutic agents are highly lipophilic, for example steroids, prostaglandins, leukotrienes, and fat-soluble vitamins. Such compounds may be advantageously administered in oil-in-water emulsion vehicles comprising a formaldehyde sulfoxylate or its pharmaceutically acceptable salts thereof as a preservative, particularly when administration will occur over an extended period. Accordingly, the present invention provides a sterile, therapeutic composition comprising a lipophilic pharmaceutical or therapeutic agent, either alone or dissolved in a water-immiscible solvent, emulsified in water, which further comprises an amount of formaldehyde sulfoxylate or its pharmaceutically acceptable salts thereof effective as a preservative.

### Example 3

Tables 3-7 illustrate the antimicrobial effectiveness of propofol formulation containing either EDTA (Diprivan®) or sodium metabisulfite. In these studies, EDTA was used at 0.005% wt, and sodium metabisulfite was used at 0.025% wt. Unpreserved propofol formulation (i.e., does not contain any antimicrobial agent) was used as a negative control.

The representative results (Tables 3-7) show that EDTA and sodium metabisulfite were highly effective in preventing the significant growth of microorganisms for at least 24 hours after adventitious, extrinsic contamination, when compared to the unpreserved propofol formulation.

In the present study, we also prepared a propofol formulation containing sodium formaldehyde sulfoxylate (formulation no. 1) and evaluated its anti-microbiological activity. Formulation no. 1 contained propofol (10 mg/mL), soybean oil (100 mg/mL), glycerin (22.5 mg/mL), egg-yolk phospholipid (12 mg/mL), and sodium formaldehyde sulfoxylate (0.5 mg/mL). In brief, a solution of sodium formaldehyde sulfoxylate in water-for-injection was prepared and pH was adjusted to 7.86 using hydrochloric acid (0.1N). Lecithin and glycerin were added, followed by propofol dissolved in soybean oil to form an oil-in-water emulsion. The pH of the oil-in-water emulsion was adjusted to 8.06 using NaOH (0.1N). The pH of the autoclaved final propofol oil-in-water emulsion was 7.15.

Tables 3-7 illustrate the antimicrobial effectiveness of propofol formulation no. 1 containing sodium formaldehyde sulfoxylate and show that the propofol formulation no. 1 was highly effective in preventing the significant growth of microorganisms for at least 24 hours after adventitious, extrinsic contamination.

TABLE 3
Comparison of Microbial Growth Retarding Activity
of Various Propofol Formulations (1% Injectable
Emulsions) Against S. aureus (ATCC 6538)

| | Viable count of Survivors | | Decrease in |
|---|---|---|---|
| | Log₁₀CFU/ml | | Survivors |
| Formulations | 0 hour | 24 hours | Log₁₀CFU/ml |
| Unpreserved | 2.15 | 2.83 | No decrease^{†} |
| Propofol | | | |
| Propofol + EDTA | 1.60 | 1.70 | No decrease^{†} |
| Propofol + Sodium | 1.60 | 1.40 | 0.20 |
| Metabisulfite | | | |
| Formulation No. 1 | 1.93 | 1.48 | 0.45 |

^{†}Viable count was indeed increased; and the increase was less than 10 fold.

TABLE 4
Comparison of Microbial Growth Retarding Activity
of Various Propofol Formulations (1% Injectable
Emulsions) Against E. coli (ATCC 8739)

| | Viable count of Survivors | | Decrease in |
|---|---|---|---|
| | Log₁₀CFU/ml | | Survivors |
| Formulations | 0 hour | 24 hours | Log₁₀CFU/ml |
| Unpreserved | 2.18 | 4.88 | No decrease^{††} |
| Propofol | | | |
| Propofol + EDTA | 1.90 | 0.90 | 1.00 |
| Propofol + Sodium | 1.90 | 0 | 1.90 |
| Metabisulfite | | | |
| Formulation No. 1 | 1.96 | 1.91 | 0.05 |

^{††}Viable count was indeed increased; and the increase was greater than 10 fold

TABLE 5
Comparison of Microbial Growth Retarding Activity
of Various Propofol Formulations (1% Injectable
Emulsions) Against P. aeruginosa (ATCC 9027)

| | Viable count of Survivors | | Decrease in |
|---|---|---|---|
| | Log₁₀CFU/ml | | Survivors |
| Formulations | 0 hour | 24 hours | Log₁₀CFU/ml |
| Unpreserved | 2.08 | 3.28 | No decrease^{††} |
| Propofol | | | |
| Propofol + EDTA | 1.5 | 0.9 | 0.6 |
| Propofol + Sodium | 1.5 | 0 | 1.5 |
| Metabisulfite | | | |
| Formulation No. 1 | 1.73 | 0 | 1.73 |

^{††}Viable count was indeed increased; and the increase was greater than 10 fold

TABLE 6
Comparison of Microbial Growth Retarding Activity
of Various Propofol Formulations (1% Injectable
Emulsions) Against C. albicans (ATCC 10231)

| | Viable count of Survivors | | Decrease in |
|---|---|---|---|
| | Log₁₀CFU/ml | | Survivors |
| Formulations | 0 hour | 24 hours | Log₁₀CFU/ml |
| Unpreserved | 2.11 | 1.56 | 0.55 |
| Propofol | | | |
| Propofol + EDTA | 1.70 | 1.70 | 0 |
| Propofol + Sodium | 1.70 | 1.70 | 0 |
| Metabisulfite | | | |
| Formulation No. 1 | 1.48 | 1.52 | No decrease^{†} |

^{†}Viable count was indeed increased; and the increase was less than 10 fold

TABLE 7
Comparison of Microbial Growth Retarding Activity
of Various Propofol Formulations (1% Injectable
Emulsion) Against A. niger (ATCC 16404)

| | Viable count of Survivors | | Decrease in |
|---|---|---|---|
| | Log₁₀CFU/ml | | Survivors |
| Formulations | 0 hour | 24 hours | Log₁₀CFU/ml |
| Unpreserved | 2.08 | 0.78 | 1.30 |
| Propofol | | | |
| Propofol + EDTA | 1.70 | 0.7 | 1.00 |
| Propofol + Sodium | 1.70 | 0.50 | 1.20 |
| Metabisulfite | | | |
| Formulation No. 1 | 1.88 | 1.79 | 0.09 |

These data clearly indicate that sodium formaldehyde sulfoxylate is effective in preventing a no more than 10-fold increase in the growth of each of the microorganisms tested over a 24 hour period.

General Process of Preparing Propofol Composition Containing Sodium Formaldehyde Sulfoxylate

The preparation of the oil-in-water emulsion of propofol consists of the following steps:
1) dissolving sodium formaldehyde sulfoxylate or its pharmaceutically acceptable salts thereof in about 50% weight water-for-injection (WFI) to form an aqueous phase in a first tank;
2) adding hydrochloric acid (q.s. to adjust the pH to 7.86) while maintaining the temperature of the aqueous phase at about 55° C.;
3) mixing egg-yolk phospholipid (lecithin) (purchased from Ferro Labs., Waukegan, Ill.) to about 30% weight WFI in a second tank while maintaining the temperature of the dispersion at about 25° C.;
4) adding glycerin (purchased from Ruger Chemicals, Irvington, N.J.) to the egg-yolk phospholipid dispersion of step (3);
5) filtering the egg-yolk phospholipid dispersion of step (4) through a 5.0 μm filter;
6) adding the filtrate of step (5) to the solution of step (2) in the first tank;
7) adding about 10% weight WFI to the compounding tank to make up the weight of aqueous phase;
8) homogenizing the aqueous phase of step (7) while maintaining the temperature of the dispersion to about 55° C.;
9) dissolving propofol (purchased from Zambon, Lonigo, Italy) in soybean oil (purchased from Croda, Edison, N.J.) to form an oil phase in a third tank while maintaining the temperature of the oil phase at about 55° C.;
10) filtering the oil phase in step (9) through a 0.45 μm filter;
11) adding the oil phase of step (10) to the aqueous phase of step (8) to form a crude emulsion in the first tank;
12) homogenizing the crude emulsion while maintaining the temperature at about 55° C.;
13) cooling the crude emulsion to about 30° C.;
14) adjust the pH of the crude emulsion in step (13) to about 8.06
15) microfluidizing the crude emulsion in step (14) to targeted globule size to form an oil-in-water emulsion;
16) filtering the oil-in-water emulsion;
17) filling and sealing the oil-in-water emulsion in a container under nitrogen; and
18) autoclaving the oil-in-water emulsion to obtain propofol formulation containing formaldehyde sulfoxylate or its pharmaceutically acceptable salts thereof.

Preferably, all steps are performed under nitrogen.

A pharmaceutically acceptable salt of formaldehyde sulfoxylate (such as sodium formaldehyde sulfoxylate) is conveniently dissolved in the aqueous phase during step (1) and remain largely unchanged in steps (1)-(2). Typically, mixing step for egg-yolk phospholipid is performed for about 20 minutes at about 250 rpm. Glycerin is added in step (4) to adjust the isotonicity. Suitable isotonic agents may be used. Homogenizing step (12) usually is performed at about 9,800 rpm for a time period sufficient to obtain optimal effective diameter of the droplets (i.e., globule size) in the oil-in-water emulsion. The diameter of the droplets is conveniently determined by using Brookhaven Multiangle Particle Sizing equipment. Typically, the diameter of the droplets in the oil-in-water emulsion is adjusted to about 200 nm.

Preferably, autoclaving is used for terminal sterilization to obtain the oil-in-water emulsion. Other suitable sterilization means may be used, such as filtration.

Preferably, the thermal lability and sensitivity to oxidation of sodium formaldehyde sulfoxylate necessitate accurate temperature control and a nitrogen or other inert gas environment in the manufacturing process.

The present procedure may be modified to prepare other compositions of the present invention by substituting other water immiscible solvents for the soybean oil, other surfactants for the egg yolk phospholipid, other acids or bases to adjust the pH instead of sodium hydroxide, and/or other tonicity modifiers for the glycerin. The procedure may also be modified to prepare other drugs in a preserved oil-in-water emulsion or those for parenteral nutrition.

Microbiological Activity

The present invention provides a sterile pharmaceutical preparation of propofol that comprises an amount of a pharmaceutically acceptable salt of formaldehyde sulfoxylate sufficient to significantly prevent the growth, or prevent no more than 10-fold increase in growth of each of S. aureus (ATCC 6538), E. coli (ATCC 8739), P. aeruginosa (ATCC 9027), C. albicans (ATCC 10231), and A. Niger (ATCC 16404). Furthermore, in the event of improper aseptic handling of the finished product leading to an accidental extrinsic contamination, the present formulation will suppress, minimize, or limit the chance of microbial growth for at least 24 hours.

The growth retarding capability of 1% propofol injectable emulsion containing formaldehyde sulfoxylate or its pharmaceutically acceptable salts thereof was evaluated using broth cultures. In brief, approximately 50-100 colony forming units (CFU) per mL of five (5) standard organisms recommended by United States Pharmacopeia (USP) for preservative efficacy tests were inoculated in each formulation. The microorganisms tested were Staphylococcus aureus (ATCC 6538), Escherichia coli (ATCC 8739), Pseudomonas aeruginosa (ATCC 9027), Candida albicans (ATCC 10231), and Aspergillus niger (ATCC 16404). All the microorganisms used in the present study (including bacterial, yeast and fungi strains) are conveniently obtained from American Tissue Cell Culture (ATCC, Manssas, Va.).

The antimicrobial activity of propofol containing a pharmaceutically acceptable salt of formaldehyde sulfoxylate was compared with propofol containing 0.005% disodium ethylenediaminetetraacetic acid (Diprivan® EDTA, trademark Zeneca), propofol containing 0.025% sodium metabisulfite, and a control propofol formulation lacking preservative (i.e., unpreserved propofol samples contained the same ingredients).

Microorganism Growth Retarding Assay

Microorganism Culture: Bacterial cultures were grown on Trypitcase Soy Agar (TSA) at 30-35° C. for 18-24 hours. C. albicans was grown on Sabouraud Dextrose Agar (SDA) at 20-25° C. for 44-52 hours. A. niger was also grown on SDA at 20-25° C. for 6-10 days or until good sporulation was obtained. Bacterial and C. albicans cultures were harvested using sterile saline test solution to obtain approximately 10⁸ CFU/mL. A. niger culture was harvested using sterile saline test solution containing 0.05% Tween 80 to obtain approximately 10⁸ CFU/mL. Bacterial and yeast saline suspension was verified by optical density at 425 nm.

Inoculation: Sample(s) of different propofol formulations were divided into equal portions in separate sterile test tubes. An aliquot of the microorganism suspensions from each species derived from TSA or SDA as cited above was inoculated aseptically into the samples to achieve approximately 50-100 CFU/mL.

Verification of reference zero-time counts was made, by introducing the same volume of microorganism suspensions into separate equivalent quantities of 0.1% peptone water for each microorganism. Zero-time counts were used as controls. Plate counts were performed to enumerate the inoculum at zero-time.

Recovery of Microorganisms: Samples were incubated for various times (e.g., 24 hours). The inoculated samples and controls were incubated at room temperature (i.e., 20-25° C.). Recovery of viable bacteria, yeast and fungi was performed at the cited time intervals by taking one (1) mL of the inoculated test material and diluting it ten-fold serially into 9 mL broth. For bacteria, a standard plate count was performed from each dilution blank with the broth.

Recovery plates were incubated at 20-25° C. for 2-3 days for bacteria and 5-7 days for yeast and fungi. Results were reported as viable count of survivors (log₁₀ CFU/mL).

The preservative was considered effective if the microbial growth was suppressed, or allowed for a no-more-than 10-fold increase in growth as compared to the zero-hour viable count (count of the microorganisms immediately following inoculation) of each of the test microorganisms.

### Example 4

We prepared a propofol formulation containing sodium formaldehyde sulfoxylate (formulation no. 2) and evaluated its anti-microbiological activity. Formulation no. 2 contained propofol (10 mg/mL), soybean oil (100 mg/mL), glycerin (22.5 mg/mL), egg-yolk phospholipid (12 mg/mL), and sodium formaldehyde sulfoxylate (0.5 mg/mL). In brief, a solution of sodium formaldehyde sulfoxylate in water-for-injection was prepared and pH was adjusted to 7.10 using hydrochloric acid (0.1N). Lecithin and glycerin were added, followed by propofol dissolved in soybean oil to form an oil-in-water emulsion. The pH of the oil-in-water emulsion was adjusted to 7.0 using NaOH (0.1N). The pH of the autoclaved final propofol oil-in-water emulsion was 6.78.

Tables 8-12 illustrate the antimicrobial effectiveness of propofol formulation no. 2 containing sodium formaldehyde sulfoxylate and show that the propofol formulation no. 2 was highly effective in preventing the significant growth of microorganisms for at least 24 hours after adventitious, extrinsic contamination.

TABLE 8
Microbial Growth Retarding Activity of Propofol Formulation
No. 2 (1% Injectable Emulsion) Against S. aureus (ATCC 6538)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 2 | 1.99 | 0 | 1.99 |

TABLE 9
Microbial Growth Retarding Activity of Propofol Formulation
No. 2 (1% Injectable Emulsion) Against E. coli (ATCC 8739)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 2 | 2.18 | 2.77 | No decrease^{†} |

^{†}Viable count was indeed increased; and the increase was less than 10 fold

TABLE 10
Microbial Growth Retarding Activity of Propofol Formulation No.
2 (1% Injectable Emulsions) Against P. aeruginosa (ATCC 9027)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 2 | 1.78 | 0 | 1.78 |

TABLE 11
Microbial Growth Retarding Activity of Propofol Formulation No.
2 (1% Injectable Emulsion) Against C. albicans (ATCC 10231)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 2 | 2.08 | 1.18 | 0.9 |

TABLE 12
Microbial Growth Retarding Activity of Propofol Formulation
No. 2 (1% Injectable Emulsion) Against A. niger (ATCC 16404)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 2 | 2.04 | 0.48 | 1.56 |

These data clearly indicate that sodium formaldehyde sulfoxylate is effective in preventing a no more than 10-fold increase in the growth of each of the microorganisms tested over a 24 hour period.

### Example 5

We prepared a propofol formulation containing sodium formaldehyde sulfoxylate (formulation no. 3) and evaluated its anti-microbiological activity. Formulation no. 3 contained propofol (10 mg/mL), soybean oil (100 mg/mL), glycerin (22.5 mg/mL), egg-yolk phospholipid (12 mg/mL), and sodium formaldehyde sulfoxylate (0.5 mg/mL). In brief, a solution of sodium formaldehyde sulfoxylate in water-for-injection was prepared and pH was adjusted to 5.0 using hydrochloric acid (0.1N). Lecithin and glycerin were added, followed by propofol dissolved in soybean oil to form an oil-in-water emulsion. The pH of the oil-in-water emulsion was adjusted to 7.02 using NaOH (0.1N). The pH of the autoclaved final propofol oil-in-water emulsion was 6.96.

Tables 13-17 illustrate the antimicrobial effectiveness of propofol formulation no. 3 containing sodium formaldehyde sulfoxylate and show that the propofol formulation no. 3 was highly effective in preventing the significant growth of microorganisms for at least 24 hours after adventitious, extrinsic contamination.

TABLE 13
Microbial Growth Retarding Activity of Propofol Formulation
No. 3 (1% Injectable Emulsion) Against S. aureus (ATCC 6538)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 3 | 1.97 | 0 | 1.97 |

TABLE 14
Microbial Growth Retarding Activity of Propofol Formulation
No. 3 (1% Injectable Emulsion) Against E. coli (ATCC 8739)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 3 | 1.93 | 2.67 | No decrease^{†} |

^{†}Viable count was indeed increased; and the increase was less than 10 fold

TABLE 15
Microbial Growth Retarding Activity of Propofol Formulation No.
3 (1% Injectable Emulsion) Against P. aeruginosa (ATCC 9027)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 3 | 1.86 | 0 | 1.86 |

TABLE 16
Microbial Growth Retarding Activity of Propofol Formulation No.
3 (1% Injectable Emulsion) Against C. albicans (ATCC 10231)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 3 | 1.92 | 1.38 | 0.54 |

TABLE 17
Microbial Growth Retarding Activity of Propofol Formulation
No. 3 (1% Injectable Emulsion) Against A. niger (ATCC 16404)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 3 | 1.89 | 0.60 | 1.29 |

These data clearly indicate that sodium formaldehyde sulfoxylate is effective in preventing a no more than 10-fold increase in the growth of each of the microorganisms tested.

### Example 6

We prepared a propofol formulation containing sodium formaldehyde sulfoxylate (formulation no. 4) and evaluated its anti-microbiological activity. Formulation no. 4 contained propofol (10 mg/mL), soybean oil (100 mg/mL), glycerin (22.5 mg/mL), egg-yolk phospholipid (12 mg/mL), and sodium formaldehyde sulfoxylate (0.5 mg/mL). In brief, a solution of sodium formaldehyde sulfoxylate in water-for-injection was prepared and pH was adjusted to 5.0 using hydrochloric acid (0.1N). Lecithin and glycerin were added, followed by propofol dissolved in soybean oil to form an oil-in-water emulsion.

The pH of the oil-in-water emulsion was adjusted to 6.18 using NaOH (0.1N). The pH of the autoclaved final propofol oil-in-water emulsion was 6.30.

Tables 18-22 illustrate the antimicrobial effectiveness of propofol formulation no. 4 containing sodium formaldehyde sulfoxylate and show that the propofol formulation no. 4 was highly effective in preventing the significant growth of microorganisms for at least 24 hours after adventitious, extrinsic contamination.

TABLE 18
Microbial Growth Retarding Activity of Propofol Formulation
No. 4 (1% Injectable Emulsion) Against S. aureus (ATCC 6538)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 4 | 1.97 | 0 | 1.97 |

TABLE 19
Microbial Growth Retarding Activity of Propofol Formulation
No. 4 (1% Injectable Emulsion) Against E. coli (ATCC 8739)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 4 | 1.93 | 0 | 1.93 |

TABLE 20
Microbial Growth Retarding Activity of Propofol Formulation No.
4 (1% Injectable Emulsion) Against P. aeruginosa (ATCC 9027)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 4 | 1.86 | 0 | 1.86 |

TABLE 21
Microbial Growth Retarding Activity of Propofol Formulation No.
4 (1% Injectable Emulsion) Against C. albicans (ATCC 10231)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 4 | 1.92 | 1.40 | 0.52 |

TABLE 22
Microbial Growth Retarding Activity of Propofol Formulation
No. 4 (1% Injectable Emulsion) Against A. niger (ATCC 16404)

| | Viable count of Survivors | | Decrease in |
|---|---|---|---|
| | Log₁₀CFU/ml | | Survivors |
| Formulation | 0 hour | 24 hours | Log₁₀CFU/ml |
| Formulation No. 4 | 1.89 | 0 | 1.89 |

These data clearly indicate that sodium formaldehyde sulfoxylate is effective in preventing a no more than 10-fold increase in the growth of each of the microorganisms tested over a 24 hour period.

### Example 7

We tested the hypothesis that formaldehyde sulfoxylate may exert its antimicrobial effects by virtue of its anti-oxidant activity (i.e., scavenging oxygen radicals). Table 23 summarizes representative studies and compares the antimicrobial effectiveness of propofol formulations containing various anti-oxidants with that of formaldehyde sulfoxylate. In these studies, nine (9) anti-oxidants were used; some represents a combination of two (2) anti-oxidants (e.g., butylated hydroxy toluene (BHT) and butylated hydroxy anisole (BHA)) at concentrations commonly used for their anti-oxidant effects. Unpreserved propofol formulation (i.e., does not contain any antimicrobial agent) was used as a negative control. The six (6) anti-oxidants were compared with the pharmaceutically acceptable salt of formaldehyde sulfoxylate.

Table 23 shows that formaldehyde sulfoxylate is highly effective in preventing the significant growth of microorganisms (including S. aureus, E. coli, P. aeruginosa, C. albicans, and A. niger) for 24 hours after adventitious, extrinsic contamination. Notably, while formaldehyde sulfoxylate is effective in preventing the growth of E. coli, none of the other tested anti-oxidants show growth retarding activity against the microorganism. Benzene sulfonic acid, thioglycerol, sodium pyrophosphate, methionine and hydroxy ethyl piperazine ethane sulfonic acid failed to inhibit the growth of P. aeruginosa. (See, Table 15). Accordingly, all the six (6) anti-oxidants at the tested concentrations fail to prevent a no more than 10-fold increase in the growth of each of S. aureus, E. coli, P. aeruginosa, and C. albicans for at least 24 hours. The present study establishes that an anti-oxidant alone cannot be effective in preventing the growth of microorganisms. However, the present study does not establish that a pharmaceutically acceptable salt of formaldehyde sulfoxylate (i.e., sodium formaldehyde sulfoxylate) does not act as an anti-oxidant for its antimicrobial activity; rather, it simply suggests that other mechanism(s) may be involved.

TABLE 23
Comparison of Microbial Growth Retarding Activity of Propofol
Compositions Containing Various Antioxidants at 24 Hours

| | Microorganisms Tested | | | | | | | | | |
|---|---|---|---|---|---|---|---|---|---|---|
| | Viable Count of Survivors Log₁₀ CFU/mL at 0 and 24 hours | | | | | | | | | |
| | S. aureus | | E. coli | | P. aeruginosa | | C. albicans | | A. niger | |
| | (ATCC 6538) | | (ATCC 8739) | | (ATCC 9027) | | (ATCC 10231) | | (ATCC 16404) | |
| | 0 hr | 24 hrs | 0 hr | 24 hrs | 0 hr | 24 hrs | 0 hr | 24 hrs | 0 hr | 24 hrs |
| Control | 2.15 | 2.83 | 2.18 | 4.88 | 2.08 | 3.28 | 2.11 | 1.56 | 2.08 | 0.78 |
| Sodium | 1.0 | 0 | 1.90 | 0 | 1.50 | 0 | 1.70 | 0 | 1.70 | 0 |
| Formaldehyde | | | | | | | | | | |
| sulfoxylate | | | | | | | | | | |
| (0.05%) | | | | | | | | | | |
| BHT | 2.00 | 2.00 | 1.90 | 5.20 | 1.80 | 0 | 1.80 | 2.00 | 1.80 | 0.80 |
| (0.00003%) | | | | | | | | | | |
| and BHA | | | | | | | | | | |
| (0.0001%) | | | | | | | | | | |
| Benzene | 2.00 | 2.20 | 1.90 | 4.10 | 1.80 | 3.30 | 1.80 | 1.90 | 1.80 | 0.90 |
| Sulfonic Acid | | | | | | | | | | |
| (0.005%) | | | | | | | | | | |
| Thioglycerol | 2.00 | 2.80 | 1.90 | 5.60 | 1.80 | 3.50 | 1.80 | 2.10 | 1.80 | 1.00 |
| (0.1%) | | | | | | | | | | |
| Sodium | 1.90 | 2.50 | 2.00 | 3.80 | 1.70 | 3.50 | 1.50 | 1.60 | 1.90 | 1.90 |
| Pyrophosphate | | | | | | | | | | |
| (0.1%) | | | | | | | | | | |
| Methionine | 1.90 | 1.90 | 2.00 | 3.80 | 1.70 | 3.80 | 1.50 | 1.80 | 1.90 | 1.80 |
| (0.1%) | | | | | | | | | | |
| Hydroxy Ethyl | 1.90 | 1.60 | 2.00 | 3.60 | 1.70 | 3.50 | 1.50 | 1.60 | 1.90 | 1.80 |
| Piperazine | | | | | | | | | | |
| Ethane | | | | | | | | | | |
| Sulfonic Acid | | | | | | | | | | |
| (0.1%) | | | | | | | | | | |

### Example 8

By way of example, the following preferred compositions of the present novel propofol formulations containing a pharmaceutically acceptable salt of formaldehyde sulfoxylate is listed as follows, without being limited thereto:

1) 1% propofol emulsion for injection:
a) about 1% weight propofol;
b) about 10% weight soybean oil;
c) about 2.25% weight glycerin;
d) about 1.2% weight egg-yolk phospholipid;
e) about 0.05% weight sodium formaldehyde sulfoxylate;
f) q.s. sodium hydroxide and hydrochloric acid; and
g) water (WFI) to 100%.

Preferably, the propofol composition of the present invention has a pH of approximately 5.0-8.0.

The disclosures of the cited publications are incorporated herein in their entireties by reference. It is to be understood, however, that the scope of the present invention is not to be limited to the above examples of compositions, and methods of manufacturing same as described above. The invention may be practiced other than as particularly described and still be within the scope of the accompanying claims.

What is claimed is:

1. A sterile, pharmaceutical composition for parenteral administration which comprises an oil-in-water emulsion in which propofol is dissolved in a water-immiscible solvent, is emulsified with water, and is stabilized by means of a surfactant, and which further comprises from about 0.03% weight to about 0.1% weight a pharmaceutically acceptable salt of formaldehyde sulfoxylate sufficient to prevent a no more than 10-fold increase in the growth of each of Staphylococcus aureus (ATCC 6538), Escherichia coli (ATCC 8739), Pseudomonas aeruginosa (ATCC 9027), and Candida albicans (ATCC 10231) for at least 24 hours as measured by a test wherein a washed suspension of each organism is added to a separate aliquot of said composition at approximately 50 colony-forming units per mL and incubated at a temperature in the range 20-25° C. and are tested for viable counts of said organisms after 24 hours.

2. The sterile, pharmaceutical composition according to claim 1, wherein the pharmaceutically acceptable salt is selected from the group consisting of sodium formaldehyde sulfoxylate and potassium formaldehyde sulfoxylate.

3. The sterile, pharmaceutical composition according to claim 1, wherein the pharmaceutically acceptable salt is sodium formaldehyde sulfoxylate.

4. The sterile, pharmaceutical composition according to claim 1, wherein the composition comprises about 0.05% weight a pharmaceutically acceptable salt of formaldehyde sulfoxylate.

5. The sterile, pharmaceutical composition according to claim 1, wherein the composition comprises about 1 to about 2% weight propofol.

6. The sterile, pharmaceutical composition according to claim 1, wherein the composition has a pH of between about 5.0 to about 8.0.

7. The sterile, pharmaceutical composition according to claim 1, wherein the composition further sufficient to prevent a no more than 10-fold increase in the growth of A. niger (ATCC 16404) for at least 24 hours as measured by a test wherein a washed suspension of each organism is added to a separate aliquot of said composition at approximately 50 colony-forming units per mL and incubated at a temperature in the range 20-25° C. and are tested for viable counts of said organism after 24 hours.

8. A sterile, pharmaceutical composition for parenteral administration, comprising by weight:
a) about 1% weight propofol;
b) about 10% weight soybean oil;
c) about 2.25% weight glycerin;
d) about 1.2% weight egg-yolk phospholipid; and
e) about 0.05% weight formaldehyde sulfoxylate.

9. A method for inducing anesthesia comprising parenteral administration of a composition which comprises an oil-in-water emulsion in which propofol is dissolved in a water-immiscible solvent, is emulsified with water, and is stabilized by means of a surfactant, and which further comprises from about 0.03% weight to about 0.1% weight a pharmaceutically acceptable salt of formaldehyde sulfoxylate sufficient to prevent a no more than 10-fold increase in the growth of each of Staphylococcus aureus (ATCC 6538), Escherichia coli (ATCC 8739), Pseudomonas aeruginosa (ATCC 9027), and Candida albicans (ATCC 10231) for at least 24 h as measured by a test wherein a washed suspension of each organism is added to a separate aliquot of said composition at approximately 50 colony-forming units per mL and incubated at a temperature in the range 20-25° C. and are tested for viable counts of said organisms after 24 hours.

10. A method of maintaining anesthesia comprising parenteral administration of a composition which comprises an oil-in-water emulsion in which propofol is dissolved in a water-immiscible solvent, is emulsified with water, and is stabilized by means of a surfactant, and which further comprises from about 0.03% weight to about 0.1% weight a pharmaceutically acceptable salt of formaldehyde sulfoxylate sufficient to prevent a no more than 10-fold increase in the growth of each of Staphylococcus aureus (ATCC 6538), Escherichia coli (ATCC 8739), Pseudomonas aeruginosa (ATCC 9027), and Candida albicans (ATCC 10231) for at least 24 hours as measured by a test wherein a washed suspension of each organism is added to a separate aliquot of said composition at approximately 50 colony-forming units per mL and incubated at a temperature in the range 20-25° C. and are tested for viable counts of said organisms after 24 hours.

11. A method of sedation comprising parenteral administration of a composition which comprises an oil-in-water emulsion in which propofol is dissolved in a water-immiscible solvent, is emulsified with water, and is stabilized by means of a surfactant, and which further comprises from about 0.03% weight to about 0.1% weight a pharmaceutically acceptable salt of formaldehyde sulfoxylate sufficient to prevent a no more than 10-fold increase in the growth of each of Staphylococcus aureus (ATCC 6538), Escherichia coli (ATCC 8739), Pseudomonas aeruginosa (ATCC 9027), and Candida albicans (ATCC 10231) for at least 24 hours as measured by a test wherein a washed suspension of each organism is added to a separate aliquot of said composition at approximately 50 colony-forming units per mL and incubated at a temperature in the range 20-25° C. and are tested for viable counts of said organisms after 24 hours.

12. A process for preparing a sterile pharmaceutical composition of propofol suitable for parenteral administration, comprising the steps of:
i) dispersing at least one surfactant selected from the group consisting of egg phosphatide, soy phosphatide, polyethylene glycol, and polyethylene glycol ester in water to form a surfactant dispersion;
ii) dissolving a pharmaceutically acceptable salt of formaldehyde sulfoxylate in water to form an aqueous solution;
iii) adding the surfactant dispersion to the aqueous solution to form a mixture;
iv) dissolving propofol in at least one water-immiscible solvent selected from the group consisting of vegetable oil, monoglyceride, diglyceride, triglyceride, and fatty acid ester to form a non-aqueous propofol solution;
v) adding the non-aqueous propofol solution to the mixture of step (iii) to form a crude oil-in-water emulsion; and
vi) sterilizing the crude oil-in-water emulsion to obtain a sterile oil-in-water emulsion of propofol.
